(19) United States
(12) Patent Application Publication
(10) Pub. No.: US 2026/0232699 A1
(43) Pub. Date: Aug. 13, 2026

(54) COMPOSITIONS OF A CANNABINOID AND A KINASE INHIBITOR FOR THE TREATMENT OF CANCER

(71) Applicant: SINOVEDA CANADA INC., Edmonton (CA)

(72) Inventors: Yun Kau TAM, Edmonton (CA); Trent BJORNDAHL, Edmonton (CA); Chih-yuan TSENG, Edmonton (CA)

(21) Appl. No.: 19/154,139

(22) PCT Filed: Feb. 8, 2024
(86) PCT No.: PCT/CA2024/050165

§ 371 Date: Aug. 6, 2025

Related U.S. Application Data
(60) Provisional application No. 63/483,840, filed on Feb. 8, 2023.

(51) Int. Cl. A61K 31/00; A61K 31/44; A61P 35/00

(52) U.S. Cl. CPC A61K 31/658; A61K 31/44; A61P 35/00

(57) ABSTRACT

A composition of a cannabinoid with a kinase inhibitor is useful for the treatment of hepatocellular carcinoma (HCC). Compositions including cannabinolic acid (CBNA) and sorafenib act synergistically against HepG2 cells, an in-vitro liver cancer model, and in ex-vivo patient derived xenografts (PDX) HCC models. The effects of all compositions disclosed are superior to sorafenib alone, a first- and second-line chemotherapeutic agent of choice for the treatment of HCC.

### FIELD

This disclosure relates to novel compositions comprising a cannabinoid and a kinase inhibitor, and methods of making and using the same to treat cancer, and hepatocellular carcinoma in particular.

### BACKGROUND OF THE INVENTION

Various cancers are treated with a kinase inhibitor which target vascular endothelial growth factor (VEGF) or vascular endothelial growth factor receptor (VEGFR). Hepatocellular carcinoma (HCC) is a particularly malignant disease with unfavorable patient outcomes. There is long unmet need in the art for an effective treatment for cancers such as HCC. Less than 35% of those diagnosed with the disease survive 5 years. This number is reduced to less than 12% if the cancer spreads to nearby tissues and less than 2% if the cancer metastasizes to other organs (Kitisin, Packiam et al. 2011). To date, no approved phytocannabinoid-derived chemotherapeutic treatment option for HCC exists. HepG2 cells, originally isolated from a 15 year-old boy in 1975, is well differentiated and characterized and has long served as a model for hepatocellular carcinoma. PDX models 575, 658, and LI-011 are patient derived Human xenograft (PDX) models in nude mice available for study through the contract research organization (CRO) Charles River.

The medicinal use of cannabis dates back to ancient times, as evidenced by gold vessels containing cannabis residue which were unearthed in Scythian tombs. Today, cannabis has been approved in the United States and Canada as an adjunct to chemotherapy to alleviate nausea and vomiting, loss of appetite, and pain (Kleckner, Kleckner et al. 2019).

The anti-cancer properties of cannabinoids and other constituents in cannabis, such as terpenes, flavonoids, etc., have been studied (Blasco-Benito, Seijo-Vila et al. 2018). In the cannabis arena, tetrahydrocannabinol (THC) and cannabidiol (CBD) have been the focus of cancer research. CBD, due to its lack of central nervous system (CNS) “side effects”, has drawn more interest.

Decarboxylated cannabinoids such as THC and CBD are known to have poor oral bioavailability and drug-like properties (Meyer, Langos et al. 2018). In addition, their low solubility and high first-pass metabolism make them poor candidates for oral delivery. For example, oral bioavailability of CBD ranges from 9-30%, which is accompanied by huge inter-individual variations in plasma profiles. In contrast, carboxylated forms of cannabinoids afford markedly increased solubility and bioavailability compared to their decarboxylated counterparts making them better drug candidates for oral administration provided they are bioactive. For example, cannabidiolic acid (CBDA) is about 25,000 times more water soluble compared to its decarboxylated counterpart, CBD (17.5 mg/ml versus 0.7 μg/ml).

An emerging therapeutic target are the agonistic or antagonistic compounds to modify heterodimer formations within numerous C-coupled protein receptors, which cannabinoid receptors are part of. This opens a plethora of new disease pathways as this superfamily of receptors comprises about 4% of the protein coding genome (Moreno, Cavic et al. 2019). New targets may provide mechanistic description of cannabinoid and/or other compound interactions.

The effects of cannabinoids can be explained by their variable binding affinities to multiple G-coupled protein heterodimeric receptors such as CB1, CB2, GPR55, and TPRV1, conveying multiple downstream pathways resulting in variable drug responsiveness (Moreno, Cavic et al. 2019). Indeed, it has been reported by Zhong (2020) that cannabinol (CBN) mediates apoptosis through the MAPK/ERK and PI3K-ATK pathways and cell cycle arrest by downregulating P21. Interestingly, Zhong's work also demonstrated a downregulation of CB2 and GPR55 receptors from CBN treatment.

Whether these effects are conserved for its more soluble acid precursor form, cannabinolic acid (CBNA), remains undetermined. Moreover, Torres, Lorente et al. (2011) demonstrated a synergistic response when THC was administered with a selective ALK inhibitor, TAE-684, indicating additive or synergistic effects can be augmented by targeting convergent pathways. Again, whether these effects could be replicated with a cannabinoid such as CBN/CBNA and a compound with convergent mechanisms of actions was unknown prior to this discovery.

### SUMMARY OF THE INVENTION

Aspects of this disclosure generally relate to synergistic anti-cancer activities of a cannabinoid and a kinase inhibitor. The cannabinoid may comprise either the carboxylated or decarboxylated form. The kinase inhibitor may comprise sorafenib or regorafenib. In one embodiment, synergistic anti-cancer activities of CBN or CBNA and sorafenib or regorafenib in the treatment of a cancer are described in this disclosure.

In one aspect, disclosed is a synergistic composition comprising CBNA and a kinase inhibitor for treatment against a cancer. In some embodiments, the molar ratio of CBNA:kinase inhibitor is between about 1:1 to about 6:1, preferably between about 2:1 to about 5:1, and most preferably about 3:1 or 4:1.

In another aspect, disclosed is an oral dosage form comprising a synergistic composition comprising CBNA and a kinase inhibitor. The oral dosage form may comprise tablets, solutions, suspensions, powder and nano-encapsulated dosage forms. In such oral dosage forms, the molar ratio of CBNA to the kinase inhibitor in the composition is between about 1:1 to about 6:1. preferably between about 2:1 to about 5:1, and most preferably about 3:1 or 4:1.

In another aspect, disclosed is a method of treating a cancer comprising administering a synergistic combination of CBNA and a kinase inhibitor to a patient in a therapeutically effective amount. The molar ratio of CBNA:kinase inhibitor in the combination may be between about 1:1 to about 6:1, preferably between about 2:1 to about 5:1, and most preferably about 3:1 or 4:1. The method may comprise administering the combination as a fixed combination or a non-fixed combination.

In some embodiments, the cancer which can be treated with the synergistic combination disclosed herein is a cancer for which sorafenib or regorafenib is effective in treating, such as hepatocellular carcinoma, renal cell carcinoma, thyroid carcinoma, metastatic colorectal cancer, or gastrointestinal stromal tumors,

### BRIEF DESCRIPTION OF THE FIGURES

FIG. 1. Chemical structures of the carboxylated form of cannabinol (cannabinolic acid (CBNA)), sorafenib, and regorafenib.

FIG. 2A. Comparisons of observed and estimated additive IC₅₀ values of different CBNA and sorafenib ratios determined in DMEM media with 1% FBS from three separate experiments (n=3). FIG. 2B. Comparisons of observed (n=1) and predicted/calculated additive IC₅₀ values of different CBNA and sorafenib ratios determined in DMEM media with 20% FBS.

FIG. 3. Histogram plots of CBNA:sorafenib vs. sorafenib IC₅₀ values (μM) in different media (DMEM with 1% FBS vs. IMDM media containing 20% FBS).

FIG. 4. HepG2 spheroids formed in 96 well, ultra-low attachment round bottom plates after 2.5 days of incubation at 37° C., 5% CO₂ in William's E media with 10% FBS.

FIG. 5. IC₅₀ value plots for sorafenib and CBNA:sorafenib 3:1 ratio against HepG2 spheroids in DMEM with 1% FBS.

FIG. 6A: IC₅₀ value plots for SCI-0502 against 3 PDX models and HepG2. 6B: IC₅₀ value plots for sorafenib against 3 PDX models and HepG2. 6C: Comparison of the IC₅₀ values calculated in FIGS. 6A and 6B. 6D: Comparison between the IC₅₀ values of the PDX models treated with SCI-0502 and sorafenib.

FIG. 7. IC₅₀ value plots for SCI-0502 and sorafenib against a 20-donor pool of normal hepatocytes (HC3_23).

FIG. 8A. In-cell western blot protein expression changes for HepG2 cells treated with sorafenib, 8B: CBNA, and 8C: SCI-0502 following a 6-hour treatment at their respective IC₅₀ value concentrations.

FIG. 9. Diagram of the determined mechanisms of action for sorafenib (C1) and CBNA (C2).

FIGS. 10A and 10B. Comparison of in-vivo immunoprecipitation results from the PDX tumor model (575) with implanted nanonails (Kibur Medical Inc.), an intra-tumoral device loaded with sorafenib, CBNA, and CBNA:sorafenib (4:1).

FIG. 11. DAB staining of Ki67 and projected cell proliferation plots for the nanonail implanted 575 PDX tumors treated with sorafenib, CBNA, and CBNA:sorafenib (4:1).

FIG. 12. Apoptotic index (%) of SCI-0502 vs. its positive controls, sorafenib and doxorubicin in a PDX (575) model loaded with the nanonail device in NCG mice.

FIG. 13. Plasma and liver concentration profiles of sorafenib in a subject who took 400 mg of sorafenib orally (Jain, Woo et al. 2011). Solid and dash lines were obtained from simulations and open symbols were taken from the 4 patients.

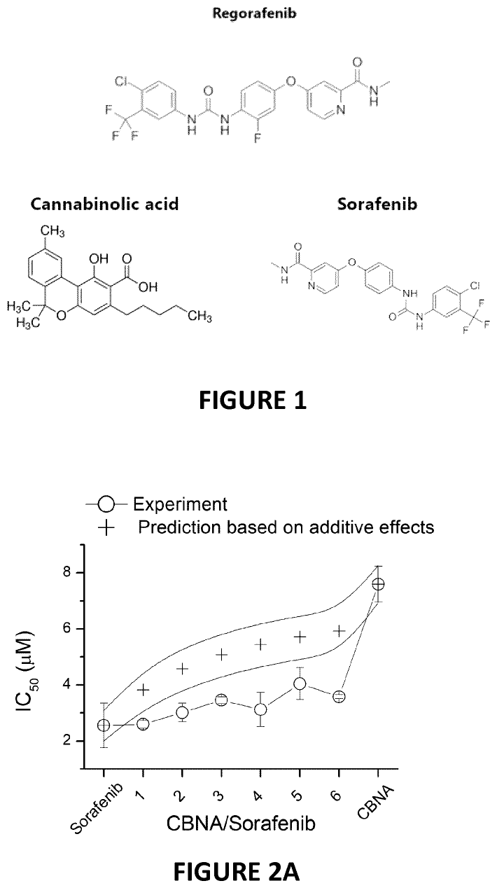

### DETAILED DESCRIPTION OF THE INVENTION

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

### Certain Terminology

The transitional terms “comprising”, “consisting essentially of”, and “consisting” are intended to connote their generally in accepted meanings in the patent vernacular; that is, (i) “comprising”, which is synonymous with “including”, “containing”, or “characterized by”, is inclusive or open-ended and does not exclude additional, unrecited elements or method steps; (ii) “consisting of” excludes any element, step, or ingredient not specified in the claim; and (iii) “consisting essentially of” limits the scope of a claim or embodiment to the specified materials or steps “and those that do not materially affect the basic and novel characteristic(s)” of the claimed invention or the embodiment. More specifically, the basic and novel characteristics relates to the ability of the method or use to provide at least one of the benefits described herein, including but not limited to the ability to improve the survivability of the human population relative to the survivability of the comparative human population described elsewhere herein. Embodiments described in terms of the phrase “comprising” (or its equivalents), also provide, as embodiments, those which are independently described in terms of “consisting of′ and “consisting essentially of”.

When a value is expressed as an approximation by use of the descriptor “about”, it will be understood that the particular value forms another embodiment. If not otherwise specified, the term “about” signifies a variance of ±10% of the associated value, but additional embodiments include those where the variance may be ±5%, ±15%, ±20%, ±25%, or ±50%, in particular the term “about” signifies a variance of ±5% or ±10% of the associated value, more in particular ±5%.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the singular forms “a,” “an,” and “the” include the plural.

As used herein, “patient” is intended to mean any animal, in particular, mammals. Thus, the methods or uses are applicable to human and nonhuman animals, although most preferably with humans. The terms “patient” and “subject” and “human” may be used interchangeably.

The terms “treat” and “treatment” refer to the treatment of a patient afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics includes, for example, improved well-being of the patient.

The term “dosage” refers to the information of the amount of the therapeutic to be taken by the subject and the frequency of the number of times the therapeutic is to be taken by the subject. The term “dose” refers to the amount or quantity of the therapeutic to be taken each time.

The term “cancer” as used herein refers to an abnormal growth of cells which tend to proliferate in an uncontrolled way and, in some cases, to metastasize (spread).

The terms “co-administration” or the like, as used herein, encompass administration of the selected therapeutic agents to a single patient, and are intended to include treatment regimens in which the agents are administered by the same or different route of administration or at the same or different time.

The term “pharmaceutical combination” as used herein, means a product that results from the mixing or combining of more than one active ingredient and includes both fixed and non-fixed combinations of the active ingredients. The term “fixed combination” means that the active ingredients, e.g., the kinase inhibitor and the cannabinoid, are both administered to a patient simultaneously in the form of a single unit or single dosage form. The term “non-fixed combination” means that the active ingredients, e.g., the kinase inhibitor and the cannabinoid, are administered to a patient as separate units or separate dosage forms, either simultaneously, concurrently or sequentially with no specific intervening time limits, wherein such administration provides safe and effective levels of the two active ingredients in the body of the human. The latter also applies to cocktail therapy, e.g., the administration of three or more active ingredients.

Described herein are compositions comprising a cannabinoid and a kinase inhibitor, exemplified by CBN or CBNA and sorafenib or regorafenib, which the inventors have determined are more effective than the kinase inhibitor (e.g. sorafenib) alone against numerous hepatocellular cancer models with a larger margin of safety when compared to a pool of normal hepatocytes. An in-vitro cell hepatocellular cancer model (HepG2), normal pool of Human hepatocytes, ex-vivo patient derived xenograft hepatocellular cancer models (575, 658, and LI-011), real time polymerase chain reaction (rtPCR) gene expression studies, and in-cell western blot protein expression studies were used to evaluate relative efficacies, and mechanisms of actions and interactions of the test compounds and their combinations thereof.

In some embodiments, the cannabinoid may comprise the decarboxylated form of the cannabinoid, such as cannabinol (CBN).

Sorafenib or regorafenib are used to treat various cancers including hepatocellular carcinoma, renal cell carcinoma, thyroid carcinoma, metastatic colorectal cancer, gastrointestinal stromal tumors. Therefore, the synergistic combinations disclosed herein may be used to treat any of these cancers, where sorafenib or regorafenib is known to be an effective treatment.

In some embodiments, sorafenib or regorafenib may be administered as a pharmaceutically acceptable salt. In a preferred embodiment, sorafenib is administered in base form. Those skilled in the art are able to determine an effective amount of a pharmaceutically acceptable salt in an amount corresponding to its base equivalent.

A salt can be prepared by for instance reacting sorafenib or regorafenib with an appropriate acid in an appropriate solvent. Acid addition salts may be formed with acids, both inorganic and organic. Examples of acid addition salts include salts formed with an acid selected from the group consisting of acetic, hydrochloric, hydriodic, phosphoric, nitric, sulphuric, citric, lactic, succinic, maleic, malic, isethionic, fumaric, benzenesulphonic, toluenesulphonic, methanesulphonic (mesylate), ethanesulphonic, naphthalenesulphonic, valeric, acetic, propanoic, butanoic, malonic, glucuronic and lactobionic acids. Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

Pharmaceutical compositions provided herein comprise an effective amount of both the cannabinoid and the kinase inhibitor. The phrases “pharmaceutical or pharmacologically acceptable” refers to molecular entities and compositions that do not produce an adverse, allergic or other unwanted reaction when administered to an animal, such as, for example, a human, as appropriate. Pharmaceutically acceptable compositions may produce tolerable side-effects. The preparation of a pharmaceutical composition that contains at least a kinase inhibitor and a cannabinoid will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by a regulatory body, such as the FDA Office of Biological Standards.

In some embodiments, the synergistic combination of a cannabinoid and a kinase inhibitor may be effective in the population at large, ie. not used in a personalized or precision medicine approach. In other embodiments, the synergistic combination of a cannabinoid and a kinase inhibitor may be effective in an identifiable subset of patients, which may be identified by a suitable test.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

Nearly all kinase inhibitors are effective when taken orally. In certain preferred embodiments, the pharmaceutical composition is administered orally to treat a cancer, such as HCC. In one embodiment, effective oral doses of the CBNA/sorafenib combination are estimated for the treatment of patients with HCC.

In certain embodiments, the actual dosage amount of a composition administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of the active compounds. In other embodiments, the active compounds may comprise between about 2% to about 75% of the weight of the unit, or between about 25% to about 60%, for example, or any range derivable therein.

In other non-limiting examples, the dosage amount of the active compounds may also comprise from about 1 milligram/kg/body weight, about 2.0 milligram/kg/body weight, about 3.0 milligram/kg/body weight, about 4.0 milligram/kg/body weight, to about 5.0 mg/kg/body weight or more per administration, and any range derivable therein. In non-limiting examples of a derivable range from the numbers listed herein, a range of about 2.5 mg/kg/body weight to about 5.5 mg/kg/body weight, can be administered, based on the numbers described above.

In one embodiment, the drug-like properties of sorafenib in humans are estimated using human pharmacokinetic data.

In one embodiment, the drug-like properties of CBNA are estimated in silico.

### EXAMPLES

These examples are provided for illustrative purposes only and not to limit the scope of the claims provided herein.

### Materials and Methods

Pure Cannabinolic acid was obtained from Cerilliant (CBNA, C-153-1ML, lot FE10222002; and Kinetochem, NGC1025, lot CBNA-RD-210826). Sorafenib was acquired from Selleckchem, (S7397, lot S739707). Sorafenib was reconstituted in DMSO to 10 mM and subsequently diluted to working concentrations. CBNA was dissolved in 95% ethanol (10 mg/ml). Compounds were added directly to media at the highest concentration (100 μg/ml) and serially diluted to subsequent working concentrations.

The designation SCI-0502 refers to a combination of CBNA and sorafenib.

### Cell Assays:

HepG2 cells were obtained from the American Type Culture Collection (ATCC). Normal hepatocyte pool of cells was acquired from Xenotech. Cryopreserved cells in DMEM containing 5% DMSO were thawed and diluted 10-fold in phenol red free DMEM media containing 10% fetal bovine serum, 100 μg/ml penicillin/streptomycin, supplemented with Glutamax™ and sodium pyruvate. Subsequently, cells were centrifuged at 20 g for 10 minutes. The cells were resuspended in the same media with added Revitacell® to a concentration of 2×10⁵ cells/ml. Ninety-six well cell culture treated flat bottom plates were seeded with 7.5×10³ cells in 100 μl and incubated at 37° C., 7.5% CO₂, >95% relative humidity for 18 hours to allow cellular adhesion after which media was replaced with like media (DMEM, IMDM, Williams E) containing 1 to 20% FBS and various test compounds (10 μg/ml to 100 ng/ml). After 48 hours (2 doublings of cells), sodium; 2-(2-methoxy-4-nitro-5-sulfonatophenyl)-3-(2-methoxy-4-nitro-5-sulfophenyl)-N-phenyltetrazol-3-ium-5-carboximidate (XTT) (300 μg/ml) was added, and cells incubated for 2 hours. Formazan production was measured at 450 nm on a Varioskan lux spectrophotometer. Non-specific absorption was measured at 660 nm and subtracted from the reference measurement at 450 nm. Experiments were performed in triplicates (n=3). Following blank subtraction, the data were averaged and fit to four-point logistic regression curves to obtain IC₅₀ values. Blank samples included equivalent solvent concentrations as a negative control. The ethanol or DMSO concentrations for blank standards reflected those used in successive cannabinoid serial dilutions. Initial studies were performed to assess single cannabinoid activity toward HepG2 cell proliferation.

### Spheroid (Solid Tumor) Assay:

HepG2 cells were thawed and reconstituted in Williams E media supplemented with Gibco hepatocellular maintenance supplement. Cells were seeded into Corning® spheroid black-walled, clear round-bottomed, ultra-low attachment, 96-well microplates at a density of 1500 cells/well. The plates were spun at 10×G for 20 minutes to congregate cells into the center of the wells. After a 60-hour incubation at 37° C., 5% CO₂, spheroids were subsequently treated with a CBNA to sorafenib ratio of 3:1 for 48 hours before Calcein-AM addition (1 μM). Spheroid vitality was measured with (excitation/emission: 508/527 nm) following a 60-minute incubation at 37° C. Experiments were run in triplicates (n=3). Following blank subtraction, the data were averaged and fit to four-point logistic regression curves to obtain IC₅₀ values.

### Normal Hepatocyte Cell Line Studies:

To evaluate the safety margin of the compounds prior to clinical trials, the toxicity of the compounds toward a pool of normal hepatocytes was investigated in-house using a hepatocyte pool obtained from Xenotech (HC3_23). Cells were thawed and plated in 96 well, black walled, gamma-irradiated plates (100 μl) after reconstitution in William's E media with Gibco hepatocellular maintenance supplement. Following an 18-hour recovery period, the media was exchanged with the same media containing 9 serial dilutions of the test compound mixtures (8 to 2000 μg/ml). Experiments were performed in triplicate. Following a 48-hour incubation period, Calcein-AM was added to provide a final concentration of 7 ng/ml to each well and allowed to react for 60 minutes before readings were acquired (excitation 494 nm, emission 517 nm). Data were fit to sigmoidal curves using four-point logistic curve fitting software and the 50% inflection points used for IC₅₀ values.

### Clonogenic Assay:

Tumor xenografts (patient-derived: 575, 685, and LI-011; as well as cell line-derived: HepG2) were passaged as subcutaneous xenografts in NMRI nu/nu mice. Once tumor volumes reached 600-1000 mm³, tumors were harvested according to FELASA and GV-SOLAS animal welfare guidelines. Tumors were mechanically disaggregated and subsequently incubated with collagenase type IV (41 U/mL), DNase I (125 U/mL), hyaluronidase type III (100 U/mL), and dispase II (1 U/mL) in RPMI 1640 medium at 37° C. for 60-120 minutes. Cells were filtered through 100 μm and 40 μm mesh size sieves and washed with the same media devoid of enzymes and stored in liquid nitrogen vapor phase.

The clonogenic assay was carried out in 96 well, ultra-low attachment plates. Frozen aliquots of tumor cells were prepared from HepG2 HCC cells and the three tumor xenografts described above. Cells were thawed in Iscove's Modified Dulbecco's Medium (IMDM), supplemented with 20% (v/v) fetal calf serum, 50 μg/ml gentamicin, 1% ethanol, and 0.4% (w/v) agar prior to use. Test wells were first layered with 100 μl of the same soft-agar medium followed by 50 μl of media with tumor cells. Following solidification, the soft-agar layer was covered with 90 μl of the same culture medium without agar. After 24 hours, 10 μl of test compounds or control medium were added and left for 14 days (continuous exposure, 100 μl drug overlay). Incubation parameters were 37° C., 7.5% CO₂, and >95% humidify. Investigational compounds including Sorafenib were serially diluted in media with 1% ethanol. Sunitinib (positive control) was serially diluted in DMSO and transferred in cell culture medium before being added to the assay plates. For efficacy determination, every 96 well plate included six vehicle-treated control wells and drug-treated wells in triplicates (Sunitinib in duplicates), and nine concentrations of test compound combinations. At the time of maximum colony formation, vital colonies were stained for 48 hours with a sterile aqueous solution of INT (2-(4-iodophenyl)-3-(4-nitrophenyl)-5-phenyltetrazolium chloride, 1 mg/mL, 25 μl/well), and colony counts are performed with a Biosys 5000 Va Bioreader with a colony diameter setting of >50 μm (area >2000 μm²). Sigmoidal concentration-response curves were fitted to the data points (test-versus-control, T/C values) obtained for each tumor model using 4 parameter non-linear curve fitting. IC₅₀ values are reported as absolute total IC₅₀ values (μM) for individual and combination therapies, being the concentration of test compound at the intersection of the concentration-response curves with T/C=50%. For calculation of mean IC₅₀ values the geometric mean is used.

### Mass Spectrometry:

HPLC-DAD/MS techniques were used to confirm concentrations and ratios of individual cannabinoids. Random samples (50 μl) were taken from the test set after 30 minutes of incubation and crashed with 150 μl of methanol to precipitate media proteins. The samples were centrifuged at 21910 RCF for 5 minutes and the supernatants were collected into 2 mL glass vial with insert. Quantitative data were acquired using an Agilent 6410 triple quadrupole mass spectrometer coupled to a 1260 series HPLC and UV detector. A thermostatic autosampler at 4° C. was used for injecting 5 μL of the samples into a Phenomenex Kinetex 5 μm XB-C18 100 Å, 250×4.6 mm column equipped with a C18 Security Guard ULTRA Cartridge at 40° C. with a flow rate of 1.00 mL/min and a gradient of 72 to 100% methanol in 45 minutes. The mobile phase A contained water with 0.05% ammonium acetate and mobile phase B consisted of methanol with 0.05% ammonium acetate. UV detection was used at 215 nm with a signal bandwidth of 4 nm. Selected ion monitoring (SIM) LC/MS analysis was performed with the following parameters at negative ion mode: The source gas temperature was set at 320° C. with a flow rate of 12 L/min, the capillary voltage maintained at −3.8 kV, dwell times were set at 200 ms with the fragment voltage set at 135 V and gas nebulizer pressure was set to 35.0 psi. Concentrations were determined by quantifying the area response of the samples against a standard calibration curve with a range from 0.025 to 10 μg/ml for the MS detector and a range from 0.5 to 15 μg/mL for the UV detector.

### Gene Expression Analysis:

Target cancer gene pathways were analyzed using a custom Taqman® array card exploring 90 unique genes associated with cancer related pathways (Kanehisa, Goto et al. 2010).

Two million HepG2 cells in IMDM media with 20% FBS, and 1% ethanol were seeded in T75 flasks and incubated at 37° C., 7.5% CO₂ until 70-80% confluent (approx. 7.5×10⁵ cells). The media were replenished with media containing test compounds at their predetermined IC₅₀ concentrations (sorafenib 12 μM, SCI-0502 12 μM, and CBNA 20 μM). Untreated cells were included as a negative control. Glyceraldehyde-3-phosphate dehydrogenase (GAPDH) was included as an endogenous control and used for normalization of the data.

Following a 6-hour incubation with the test compounds, the RNA was extracted using Invitrogen's Dynabead mRNA DIRECT kit using the recommended protocol to provide 50 μL of a purified mRNA solution. Fifty μL of RT master mix was mixed into the mRNA solution and the mixture left to react at 37° C. for 60 minutes. Reverse transcriptase inactivation was performed at 95° C. for 5 minutes and the resulting cDNA samples were kept at 4° C. until use. Fifty-five microliters of TaqMan Fast Advance master mix were added to an equal amount of cDNA sample containing 1 μg/100 μl of cDNA, which was loaded into each card reservoir. The cards were centrifuged twice at 1200 RPM for 3 minutes in a Legend XFR centrifuge and the rtPCR reaction was conducted in a QuantStudio7 PCR instrument using the recommended settings with 40 amplification cycles. Data processing was done using ThermoFisher's Connect™ application.

### In-Cell Western-Blot Analysis:

Protein expression was assessed by seeding 1000 HepG2 cells/well from cryogenically frozen stocks into 384 well optical bottom black culture plates from Thermo-Scientific (item 142761) in IMDM media with 20% FBS and 1% ethanol. After a 48-hour recovery, the media was replaced with the same media containing either Sorafenib 12 μM, CBNA 20 μM, or SCI-0502 12 μM). After six hours treatment, the cells were fixed with 4% formaldehyde for 15 minutes, permeated with 0.1% triton-x100 for 10 minutes, and left in blocking buffer (phosphate buffered saline (PBS) with 3% bovine serum albumin (BSA)) overnight at 4 degrees Celsius. The following day, the cells were rinsed with PBS, incubated with 25 μl PBS containing primary antibody (Table 1) for 3 hours at 25° C. Subsequently, the primary antibody was removed, cells were rinsed twice with PBS, and incubated with 25 μl of PBS containing the secondary antibody (Rabbit: Alexa Fluor 790 donkey anti-rabbit IgG (H+L) Lot 2409042; Mouse: Alexa Fluor 790 donkey anti-mouse IgG (H+L) Lot 2300923) at a concentration of 1:2000. Draq5 (1:2000) was added for normalization of data. After an hour incubation in the secondary antibody, the cells were rinsed with PBS and readings at 700 and 800 nm were acquired on a Licor Odyssey fluorescence imager with a resolution of 21 μm. Analysis was subsequently performed with Empiria Studio 2.1. A total of 5 samples were collected per antibody.

TABLE 1
List of primary antibodies selected for Western-Blot analysis.

| | ThermoFisher | | Primary |
|---|---|---|---|
| Primary | Scientific | Secondary | Antibody |
| Antibody | SKU # | Antibody | Ratio |
| ERBB2 (HER2) | MA514509 | Rabbit | 1 to 250 |
| VEGA | 22341-1-AP | Rabbit | 1 to 50 |
| P53 | MA515244 | Mouse | 1 to 100 |
| C-Met | PA532357 | Rabbit | 1 to 100 |
| C-Met-PO4 | 710792 | Rabbit | 1 to 250 |
| MEK2 | PA529291 | Rabbit | 1 to 500 |
| MEK2-PO4 | 44454G | Rabbit | 1 to 250 |
| PIK3CA | MA517149 | Mouse | 1 to 200 |
| PTEN | MA512278 | Mouse | 1 to 50 |
| PTEN-PO4 | 441066G | Rabbit | 1 to 250 |
| C-MYC | 132500 | Rabbit | 1 to 100 |
| DVL2 | PA529244 | Rabbit | 1 to 100 |
| TEAD2 | PA566833 | Rabbit | 1 to 100 |
| CUL2 | 700179 | Rabbit | 1 to 250 |

### In Vivo PDX Model Assessments:

Drug candidates were mixed with FITC-labeled PEG-5000 with a milligram ratio of 2:8 in a suitable solvent and lyophilized to dryness. Samples of the resultant mixtures (10 mg) were loaded into nanonails (Kibur Medical Inc.), a device invented to test multiple samples in a single tumor. The dimension of the cylindrical device is 4 mm×820 μm containing 18 reservoirs 200 μm wide and 250 μm deep. The loaded devices were kept at −80° C. until tumor implant. NCG mice (female, 8-12 weeks old) from Charles River were injected subcutaneously with 100 μL of LIXF-575 cells per mouse. The body weight of the mice and caliper measurements of the tumors were performed every 3 days until the tumors reached 300-400 mm³ (about 12 weeks). Nine mice were used for intra-tumoral implantation. Nanonail units were implanted directly into tumor tissue using a 23-gauge tapered metal needle (Electron Microscopy Science and left for 72 hours upon which the mice were euthanized and nanonail units and tissue material recovered for immunofluorescence studies. Tumors containing nanonails were excised and preserved in 10% formalin at room temperature for 24 hours. The tissues were embedded into paraffin, snap frozen, and 8 μm orthogonal microtome sections were mounted onto slides for staining. Fifteen primary antibodies (Table 2) were selected to validate previous western blot data in addition to nine immunological targets along with Ki67 and caspase 3 for calculation of apoptotic indices. Staining was performed following permeation with 1% sodium dodecyl sulfate for 15 minutes with 1:500 to 1:2000 ratios of primary antibodies in PBS at 4 Celsius for 24 hours followed by rinsing and incubation with fluorescent labelled secondary antibodies (1:1000) overnight at 4 Celsius prior to imaging and statistical analysis (Ahn, Ferland et al. 2021).

TABLE 2
Along with nine immunological targets, fifteen
in vivo targets from PDX 575 were selected to
validate the pathway response obtained in vitro.

| Target | Process/Pathway |
|---|---|
| HER2 | Receptor |
| MET | Receptor/Hepatic growth factor |
| MAP2K | Ras/RAF/MEK |
| PTEN | ATK pathway |
| PI3K | ATK pathway |
| AKT | ATK pathway |
| SMAD2/3 | TGF signaling |
| CC3 | Apoptosis |
| Ki67 | Cell Proliferation |
| DVL2 | Wnt/cell adhesion/metastasis |
| Frizzled | Receptor/Wnt/cell adhesion/metastasis |
| WWTR1 | HIPPO |
| MYC | Cell cycle/TGFb |
| Cyclin E | Cell cycle/TGFb |
| SMO | Hedge Hog |
| FoxP3 | Immune system |
| CD8 | Immune system |
| CD3 | Immune system |
| CSF1R | Immune system |
| MHC-II | Immune system |
| CD11b | Immune system |
| Arginase-1 | Immune system |
| F4 80 | Immune system |
| CD45 | Immune system |

### Example 1

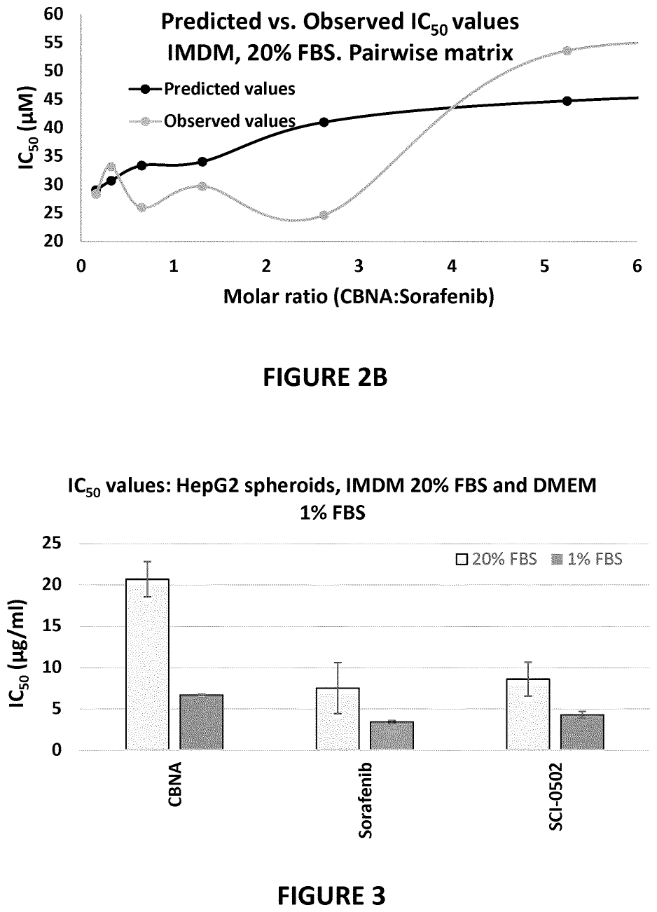

The objectives of this example are to, first, establish the procedure of a two-component interaction study; and second, establish a method of analysis to identify potential interactions among the two components/compounds. For these interaction assays, experiments were set up in 96 well plates. Serial dilutions were performed to obtain total concentrations ranging from 50 μM to 25 nM for various CBNA:sorafenib ratios. Following 48 hours of incubation, the IC₅₀ values of various mixtures were obtained and compared with the pure compound's IC₅₀ values and predicted/calculated IC₅₀ values for the various mixtures. The comparative plots of these data are shown in FIGS. 2A and 2B. FIG. 2A shows the collective results from three iterations of the experiment, where the predicted data displays a window of expected values based on the variation of individual IC₅₀ values obtained from the pure compounds. In each experiment, the observed/experimental IC₅₀ values fell below the window of expected results assuming an additive response from the two compounds. These data suggest that CBNA and sorafenib act synergistically. Moreover, the potential synergism was observed for all the various ratios explored. A more detailed explanation of data processing is outlined in the data analysis section of this example.

### Data Analysis:

Samples from the serial dilutions were analyzed by LC-MS/MS to orthogonally confirm concentrations. Ratios of the two components were organized to project IC₅₀ values for each unique concentration ratio within the data array. The Hill equation was used to model dose response relations.

$$y = {y}_{min} + \frac{{y}_{max} - {y}_{min}}{1 + {( d / {d}_{m} )}^{m}} , \qquad ( 1 )$$

where yₘᵢₙ and yₘₐₓ are minimal and maximal responses respectively, d is the test dose, dₘ is the dose that generates 50% of responses (IC₅₀) and m is the Hill coefficient, which indicates the degree of interactions between targets and chemicals and could be any values. Furthermore, following Chou's unified theory for multi-compound interaction analysis, the IC₅₀ value of a mixture of compounds that do not interact with each other can be written as a function of IC₅₀ of each compound and concentration ratios as,

$$I C 5 {0}_{a d d} = \frac{1 + {\sum }_{i = 2}^{N} {r}_{1 i}}{\frac{1}{{IC}_{{50}_{1}}} + {\sum }_{i = 2}^{N} \frac{{r}_{1 i}}{{IC}_{{50}_{i}}}} \qquad ( 2 )$$

where r₁ᵢ=dᵢ/d₁ concentration ratio of compound i to compound 1. Given this value and IC₅₀ of mixtures, IC50ₑₓₚ, three possible categories of interactions are obtained:

$$Δ IC 50 = IC {50}_{exp} - IC {50}_{add} \begin{matrix}\begin{matrix}> \\ = 0\end{matrix} \\ <\end{matrix} , \begin{matrix}\begin{matrix}Synergism < Antagonism or Antagonism only \\ Synergism \sim Antagonism or Additivity only\end{matrix} \\ Synergism > Antagonism or Synergism only\end{matrix} \qquad ( 3 )$$

To assess potential interactions, the IC₅₀ values for individual cannabinoids in the control lanes were calculated and used to predict and IC₅₀ value for the summation of their individual components.

These experiments were performed in media containing only 1% FBS and matrix effects have been observed between cannabinoids, including CBNA, and FBS components (FIG. 3). Thus, reproduction of the results was sought out in media containing 20% FBS. Indeed, an increase in IC₅₀ values for both sorafenib and CBNA was observed (FIG. 2B), accompanied by a loss of synergistic activity at higher CBNA ratios. However, additive/synergistic effects were still observed when the ratio was kept below 4:1. These results are reflected by a direct interaction of the compounds and components of the media used in the in-vitro.

### Example 2

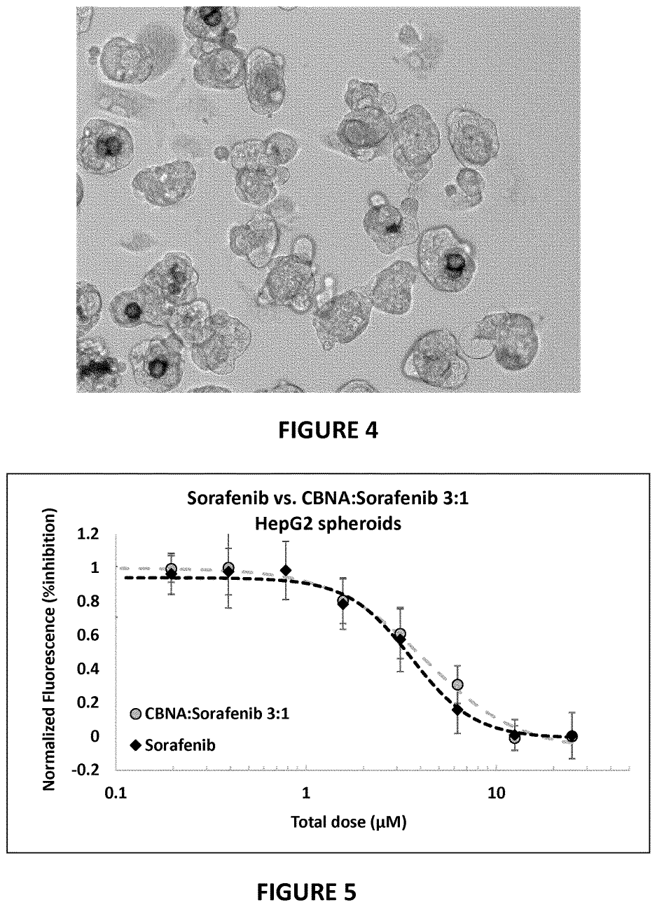

The pairwise assay results in both 1 and 20% FBS (FIGS. 2A and 2B), indicate ratios of CBNA:sorafenib between 1:1 to 4:1 present synergistic activity. To further validate these findings a solid three-dimensional spheroid/tumoroid model of HepG2 with a CBNA:sorafenib molar ratio of 3 to 1 was chosen for investigation. Due to the diffusion limitations introduced with these models, a better understanding of how the therapy will perform within a more relevant biological context can be obtained. Examples of the spheroids used in the assay are shown in FIG. 4. The dose response data was obtained using Calcein-AM fluorescence (FIG. 5). While the data reflect an additive response in comparison to the synergism observed in FIGS. 2A and 2B, it is noteworthy that the similar response with the CBNA:sorafenib mixture contained only ¼ the dose of sorafenib. These data suggest an overlap and complementary mechanisms of action between the two components may exist and dominate the dose response data observed.

### Example 3

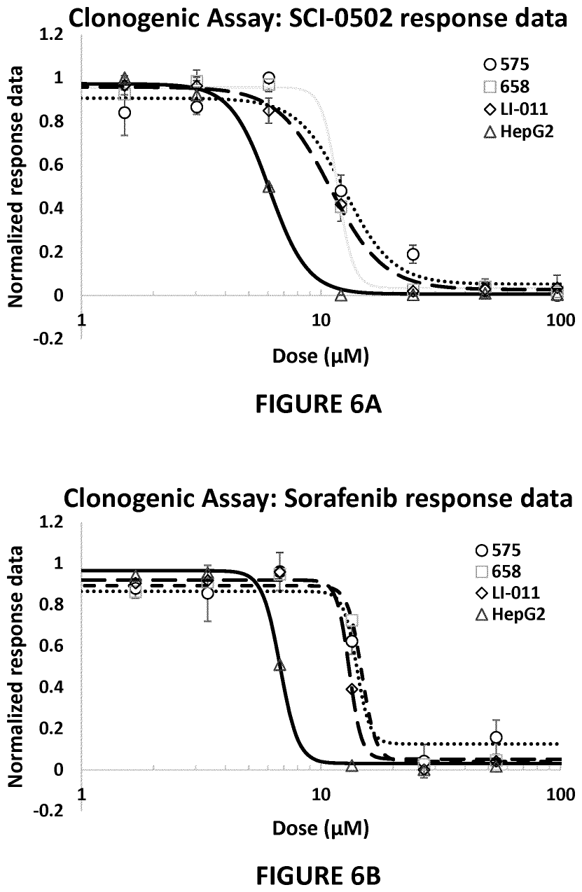

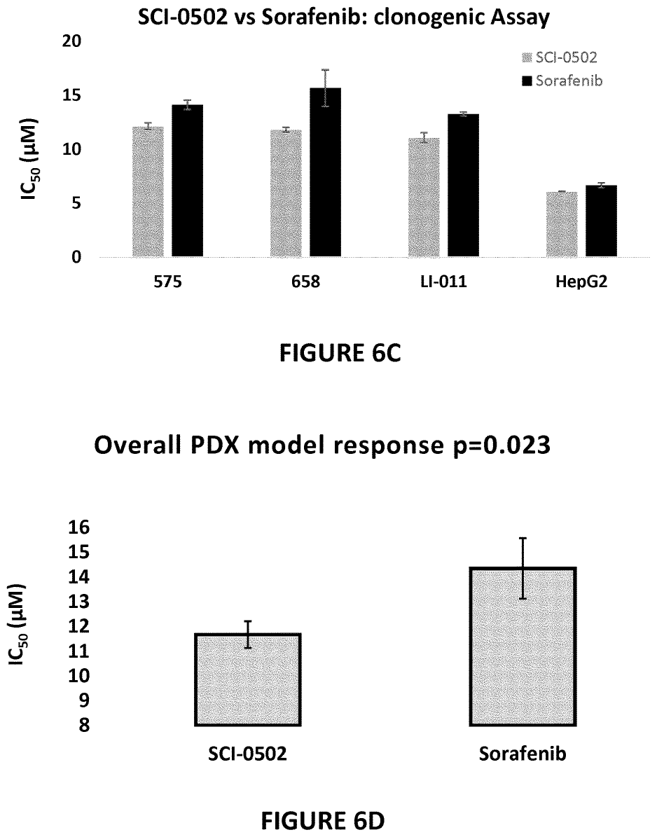

The combination candidates described in this invention were designed to disrupt multiple oncogenic pathways, which are more effective in patients with heterogenous onco-genetic landscape. The hypothesis is that therapies targeting key pathways are more effective than single target therapies. This hypothesis is tested in the combination therapies described in this invention. In order to examine this hypothesis, the efficacy towards the 575, 658, and LI-011 HCC PDX models with diverse genetic makeup and the HepG2 HCC cell line (FIGS. 6A to 6C) were compared. Consistent with the hypothesis, a better response was demonstrated for SCI-0502 compared to sorafenib (FIG. 6C) for all the PDX models explored. Additionally, despite the genetic heterogeneity of the models chosen, the efficacy (IC₅₀ values) of SCI-0502 for the three HCC PDX models were remarkably consistent. A comparison between SCI-0502 and sorafenib for all PDX models examined revealed an improvement of almost 20% in IC₅₀ values (FIG. 6D; 11.66 μM vs 14.34 μM, P=0.023). SCI-0502 proved efficacious against a variety of hepatocellular cancer cell lines with a better response compared to sorafenib, thus making it a viable candidate for further development in the treatment of hepatocellular carcinoma.

### Example 4

The objective of this example is to explore the selectivity the compounds might have toward cancer cells by performing a qualitative assessment between the IC₅₀ values observed for HepG2 hepatocellular carcinoma cells and normal hepatocytes. This study is qualitative in nature given that the two cell lines have different recommended media types with varying amounts of FBS and the FBS concentration directly impacts the IC₅₀ values obtained (FIG. 3).

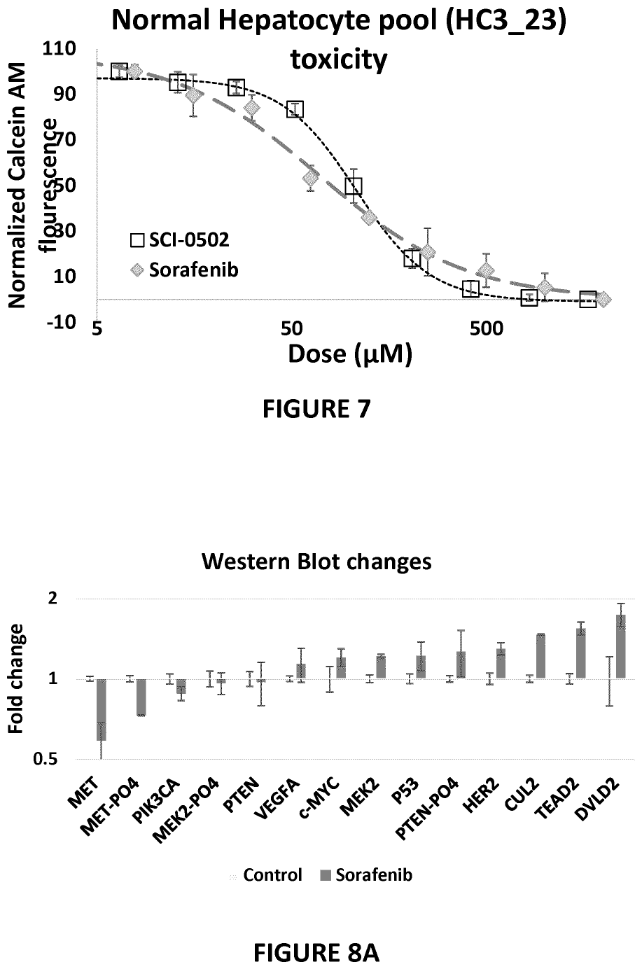

Despite these limitations, the authors were interested in preliminary data that might indicate better tolerance of the combination therapy over that of sorafenib. Sorafenib is a general multi-serine/threonine kinase inhibitor with many side effects including bleeding, breathing difficulty and skin blistering. CBNA, on the other hand, is a cannabinoid with presumed low toxicity. An investigation on the dose response comparing a 4:1 ratio of CBNA:sorafenib versus sorafenib toward HepG2 hepatocellular carcinoma and a pool of normal hepatocytes (HC3_23) was undertaken to better understand if the combination therapy had better tolerance over sorafenib monotherapy. Presumably, a lower dose of sorafenib would produce a lowered response and possibly a better tolerance. However, given the additive response observed in the pairwise matrix study this was unclear. The results from this study were compared with IC₅₀ value data obtained from previous XTT experiments performed on HepG2 cells (DMEM media with 20% FBS, 48 hour compound treatment at 37° C., 5% CO₂). These data revealed that the IC₅₀ values were 7 times higher for sorafenib and 11 times higher for SCI-0502 than those observed for HepG2 cancer cells (FIG. 7, Table 2). The IC₅₀ value of SCI-0502 was roughly 1.6 times higher than that of sorafenib in normal cells suggesting improved selectivity over sorafenib.

TABLE 3
Comparison of IC₅₀ values between sorafenib and SCI-0502 for both
HepG2 HCC cells and a pool of normal hepatocytes (HC3_23).

| | |
|---|---|
| IC₅₀ (μg/ml) | Fold difference |

| | HepG2 | HC3_23 | |
|---|---|---|---|
| SCI-0502 | 9.6 | 107.1 | 11.2 |
| Sorafenib | 9.8 | 70.1 | 7.1 |

### Example 5

The objective of this study is to understand the mechanisms of action of CBNA and sorafenib explored in this disclosure and the optimal effective ratio of these compounds against the proliferation of HepG2 cells in-vivo.

Assessment of cancer gene pathways was carried out using custom designed TaqMan array cards exploring 90 genes linked to numerous cancer related pathways. These pathways include top level G-coupled protein receptors and downstream proteins and enzymes linked to apoptosis, autophagy, cell cycle arrest, and pathways perturbed by sorafenib and CBNA discussed in this disclosure. These ΔΔCt data are shown in Table 3. Of the 90 genes explored, over ⅓ (37 genes) displayed significant changes in their Ct values. While sorafenib presented with the largest number of genes affected, the data for CBNA showed many of the same genes implicating the overlapping pathways were affected for both monotherapies.

Comparison of the CBNA, sorafenib and SCI-0502 against the negative control yielded results consistent with previous reports in the literature in addition to new findings. Indeed, this disclosure confirmed that sorafenib alters several MAP kinases and PI3K pathway genes, but also numerous genes involved with cellular processes such as angiogenesis, cellular adhesion, and cell cycle arrest.

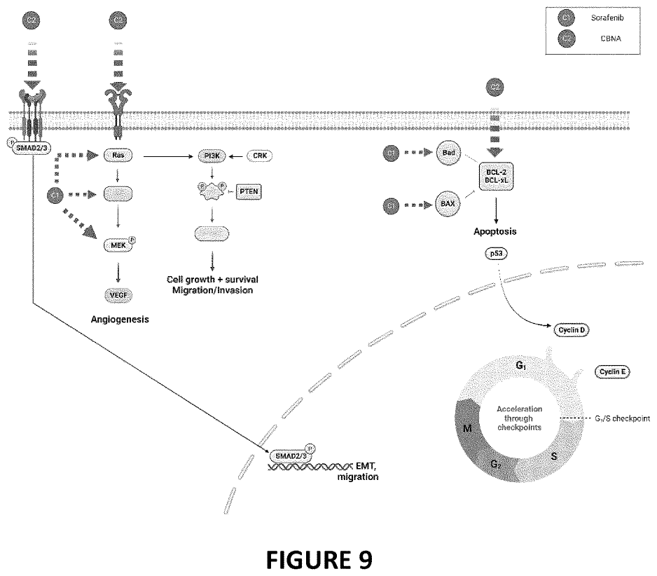

We observed that CBNA like CBN works through the MAPK/PI3K pathways (Zhong 2020). However, our data further indicate this mechanism is also mediated through PTEN in addition to MAPK/Ras control alone. These data indicate that the two compounds affect Ras/Raf/MEK pathways to alter AKT through PI3K in addition to CBNA affecting AKT via PTEN and PI3K directly through CRK. These convergent mechanisms likely result in the additive anti-oncogenic responses observed. In addition to this mechanism, convergent pathways affecting apoptosis are also observed. This directly affects cell cycle processes (via BCL2/BCL-xl) likely through the MAP kinase pathway, in which both compounds interact. These effects are outlined in FIG. 9. Many of the pathways outlined in this diagram are maintained with the treatment of SCI-0502 for the 6 hour timepoint examined (Table 3). In addition, we also observed (unreported) activation of TGF signaling and SMAD gene transcription factor, which participate in metastatic pathways and catenin gene expression (CTNNB1). Within the 6 hour timeframe of this experiment, no influence on either CB1 or CB2 receptors was observed. However, an 8-fold downregulation of the ERBB2/HER2 receptor was observed for sorafenib and CBNA but a more than 16 fold reduction was seen for the combination therapy SCI-0502. It remains unclear from these data if CBNA binds directly to these receptors, or they affect their expression through feedback mechanisms. Regardless, ERBB2 is an important target in its own right as its overexpression has not only been observed in a number of other cancer types including breast, ovarian, stomach, and lung but also targeted by other currently FDA approved therapies such as trastuzumab or Herceptin® marketed by Genentech.

TABLE 4
rtPCR of genes presenting changes in Ct values relative to untreated cells.
ΔΔCₜ changes relative to negative control.

| | | | Sorafenib | CBNA | SCI-0502 |
|---|---|---|---|---|---|
| Pathways | MAP kinase | ERBB2 | 2.729 | 3.097 | 4.225 |
| | | MAP1LC3A | −1.658 | −1.26 | |
| | | MAP3K1 | −2.162 | | |
| | | MAP2K2 | −1.241 | −1.607 | −1.906 |
| | | MAPK8 | −1.245 | | |
| | | RASA1 | −2.013 | −1.904 | |
| | PI3K pathway | CRK | −3.778 | | |
| | | PIK3C2A | −2.308 | | |
| | | PIK3CA | −1.501 | −1.639 | |
| | | PTEN | −1.165 | −1.446 | |
| | JAK-STAT | STAT1 | −1.284 | | |
| | Wnt signaling | GSK3B | −1.105 | | |
| | | ROCK1 | −1.495 | | |
| | Ca Signaling | CAMK1 | −1.249 | | 0.994 |
| | | ELK1 | | | |
| | HIPPO | TEAD2 | −1.447 | | |
| | TGF signaling | SMAD2 | −1.348 | | |
| | | SMAD3 | | 2.577 | 1.218 |
| | Hedge Hog | SMO | −1.125 | | 1.652 |
| | signaling | SUFU | −1.263 | | |
| Cell Process | Cell Cycle | BECN1 | −1.627 | −1.7 | |
| | | BAD | −1.712 | | |
| | | BAX | −1.835 | −1.062 | |
| | | BCL2L1 | −1.67 | | |
| | | CDK1 | | 1.051 | 1.247 |
| | | CDK2 | −1.072 | 1.102 | |
| | | CDK4 | −1.315 | | |
| | | CDK6 | −1.273 | | |
| | | CCND1 | | | −2.156 |
| | | CCND2 | −2.012 | −1.539 | |
| | | CCNE1 | | −2.445 | |
| | | MAX | −1.278 | | |
| | | MNT | −1.155 | −1.868 | |
| | | PAK3 | | | 1.233 |
| | Cellular Adhesion | CTNNB1 | −1.355 | | |
| | Angiogenesis | VEGFA | −2.101 | −2.861 | −1.172 |
| | Drug Resistance | MITF | −2.782 | −2.367 | |
| | Apoptosis | TP53 | | | −1.569 |

### Example 6

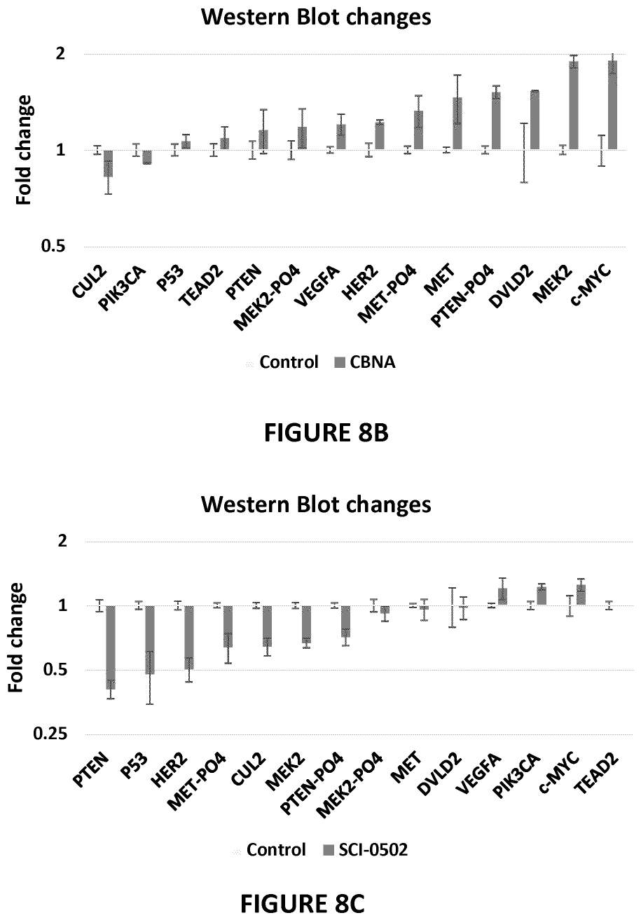

Orthogonal validation of the disclosed PCR changes was performed by in-cell western blot analysis to examine if expected changes in the protein expression correlates to the observed gene expression differences observed within the same time frame (6 hours). These data are presented in FIGS. 8A to 8C. Fourteen targets representing eleven proteins involved in pathways identified from the PCR data were chosen. Three of these proteins were evaluated in both their phosphorylated (active) and non-phosphorylated forms. These data confirm the MAPK/PI3K pathway gene expression changes observed in the PCR study. Specifically, SCI-0502 exposure to HepG2 cells affects AKT pathway through PIK3CA and PTEN regulator proteins. Moreover, confirmation of the HER2/ERBB2 gene expression is maintained. Activation of the MAP kinase protein (MEK2) has been shown to occur through ERBB2 transduction in breast cancer. This link is clearly maintained and perturbed in the hepatocellular carcinoma cell line, HepG2.

Overlap of metabolic pathways are observed in both rtPCR and western blot studies with the treatment of sorafenib and CBNA on HepG2 Cells. The synergistic effects observed in the in-vitro and ex-vivo assays performed are likely a result from pathway augmentation resulting from their convergence. While sorafenib acts directly on many of the kinase proteins involved in these pathways, CBNA is known to work through cell surface receptors. The intrinsic and extrinsic perturbations of the common pathways appear to result in synergism.

### Example 7

The objective of this example is to evaluate the predictability of in vivo mechanisms of action derived from a patient derived xenograft (PDX)

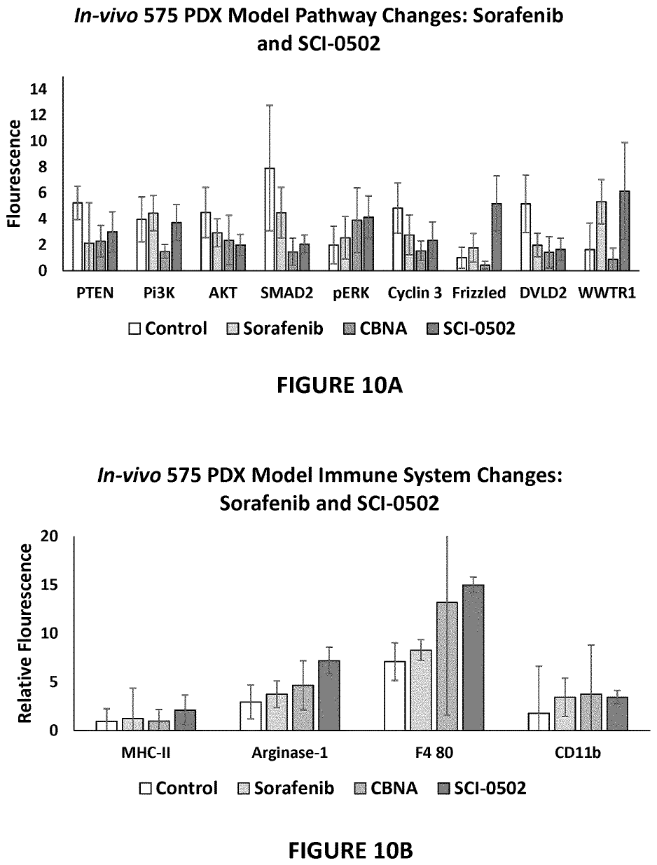

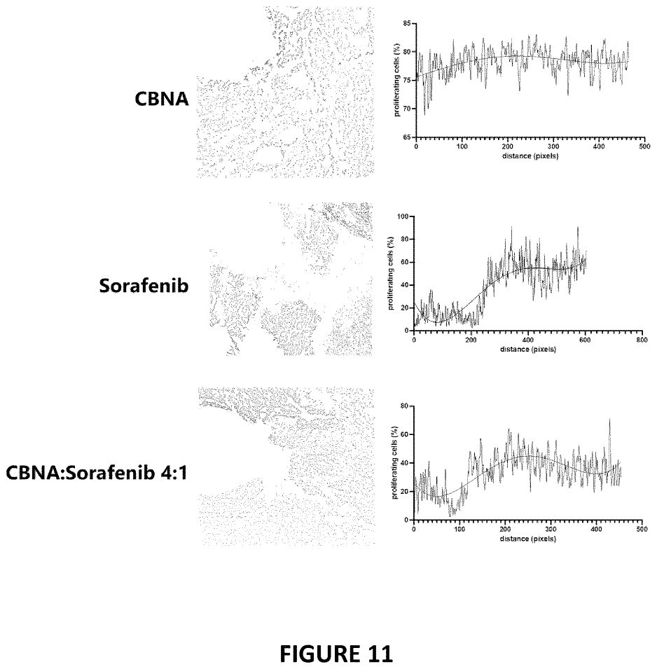

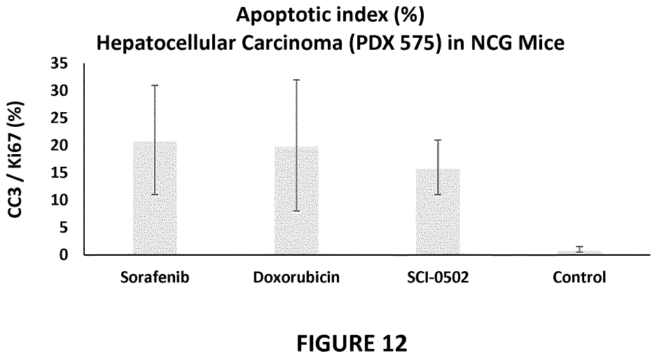

In vivo validation experiments were performed in NCG mice implanted hepatocellular carcinoma cells (575) to produce tumor xenografts and treated with drug loaded nanonails as described in [65]. These data validate previous in vitro results confirming efficacy, mechanisms of action, in addition to identifying preliminary immune system activation. The data shown in FIGS. 10A and 10B confirm the involvement of the PI3K/AKT pathway as the mechanism of action for CBNA. In addition to this, we further validated our findings that the Wnt and HIPPO pathways are affected with CBNA treatment. The activation of these pathways has been linked to metastasis. Additionally, we confirmed the involvement of macrophage activity in cleaning up the apoptotic cell debris by the positive identification of the F4/80 cell marker in tumor tissue proximal to the nanonail device (FIG. 10B). Not only do these data validate the mechanisms previously identified, but also confirm superior activity of the combination candidates within an animal study. FIG. 11 further demonstrates the safety of CBNA, in which cell proliferation is not significantly affected with its treatment alone, however, its inclusion with sorafenib still results in increased tumor death with only ⅕ the total dose compared to sorafenib alone.

The disclosure described in this invention illustrates synergism with CBNA and sorafenib. The mechanisms of action derived from HepG2 in vitro can be extrapolated to a scalable human PDX model in vivo, which is significant in the development of combination cancer candidates.

### Example 8

The objective of this example is to estimate appropriate clinical doses and the route of administration and dosages of SCI-0502.

To better estimate first in human (FIH) dose of SCI-0502, the whole body physiologically based pharmacokinetic (PBPK) model was used to simulate the temporal changes of the concentrations of sorafenib and CBNA in human. PK-Sim® version 11.2 from Open Systems Pharmacology Suite were used for the simulation (Lippert, Burghaus et al. 2019). The in-silico estimation of the pharmacokinetic parameters of CBNA and sorafenib is presented in Table 5. This set of parameters were used as initial settings for further simulations.

TABLE 5
In silico estimation of the pharmacokinetic
parameters of CBNA and Sorafenib

| Pharmacokinetic | | |
|---|---|---|
| parameters | CBNA | Sorafenib |
| F | 0.92 | 0.38-0.49[1] |
| F_{g} | 1 | 0.4-0.53 |
| Fₗ | 0.92 | 0.93-0.95 |
| Solubility, μg/ml | 10 | 3.3 [1] |
| FaSSIF | | |
| Cl_{TB}, L/min | 0.081 | 0.051-0.066[3] |
| Cl_{H}, L/min | ~0.081 | ~0.051-0.066[3] |
| Clᵣ, L/min | 0 | NA |
| V_{d, ss} (L/kg) | 2.82 | 1.01-1.3[3] |
| t½, hours | 30.08 | 14.09-18.23[3] |

| | Pₜₚ | V_{d} (L/kg) | Pₜₚ | V_{d} (L/kg) |
|---|---|---|---|---|
| Liver | 5.07 | 0.13 | 4.84 | 0.13 |
| Lung | 0.68 | 0.005 | 0.65 | 0.005 |
| Gut | 6.41 | 0.1 | 6.12 | 0.11 |
| Kidney | 3.06 | 0.013 | 2.92 | 0.013 |
| Muscle | 3.1 | 1.19 | 2.96 | 1.24 |
| Fat | 1.37 | 0.3 | 2.53 | 0.16 |

F: oral bioavailability; F_{g}: fraction of dose absorbed from the gut; Fₗ: absorbed fraction escaped the liver; FaSSIF: fasted simulated intestinal fluid; Cl_{TB}: Total body clearance; Cl_{H}: Hepatic clearance; Clᵣ: renal clearance; V_{d, ss}: Volume of distribution at steady state; t½: Half-life; V_{d}: Volume distribution of organ; Pₜₚ: tissue/plasma partition coefficient.

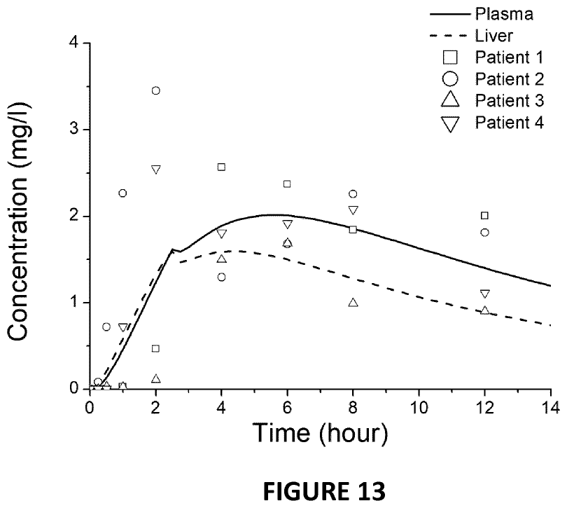

Clinical data for sorafenib from Jain, Woo et al. (2011) were utilized for the modeling if the pharmacokinetics of sorafenib. The dosage of sorafenib 400 mg twice daily was used for the simulation. The pharmacokinetic features of sorafenib in humans are enterohepatic recycling (EHC), and the drug is a substrate for canalicular efflux transporter, multidrug resistance protein 2 (MRP2). These parameters were incorporated into the simulations. In addition, parameters including passive permeability into intracellular space of organs, intestinal permeability, hepatic clearance and dissolution rate were optimized to reproduce clinical observations (Table 6 and FIG. 13). This optimization established the PBPK model for sorafenib, which describes patient plasma profiles appropriately. Because there is no clinical data available for CBNA, the PBPK model was optimized to produce results using the ADMET predictor.

TABLE 6
Pharmacokinetic parameters of sorafenib

| | Simulation | Clinical study |
|---|---|---|
| Total body clearance (l/h/kg) | 0.11 | 0.11 |
| Volume of distribution (l/kg) | 1.26 | 2.66 |
| Half life (h) | 8.63 | NA |
| Tₘₐₓ (h) | 5.5 | 2-9.5 |
| AUC₀₋₂₄ (mg*h/l) | 29.44 | NA |
| AUC₀₋₂₄ @ Liver (mg*h/l) | 21.0 | NA |

Tmax: Time to reach maximum concentration
AUC₀₋₂₄: area under concentration curve in plasma from time 0 to 24 hours
AUC_{0-24 @ Liver}: area under concentration curve in Liver tissue from time 0 to 24 hours.

Sorafenib is marketed as an oral drug, CBNA has a higher oral bioavailability than sorafenib, additionally, the hepatic clearance values between CBNA and sorafenib are comparable (Table 5), hence, it is concluded that CBNA can also be taken orally.

The ultimate objective of this example is to estimate dosages of SCI-0502 in humans which could be clinically effective. Therefore, the goal is to come up with effective doses of SCI-0502 which will achieve synergistic ratios at the site of action, the liver. AUC ratios of CBNA to sorafenib in the range of 1:1 to 6:1 in the liver were used as targets. The two PBPK models, CBNA and sorafenib, were used to calculate dosages of SCI-0502, assuming a patient who is Caucasian male, and weighing 73 kg. The dosages were administered with a meal. Simulation results are shown in Table 7.

TABLE 7
Combination dosages of sorafenib and CBNA providing equivalent or
better efficacy than 200 mg or 400 mg oral doses of sorafenib.

| Sorafenib Equivalent, 200 mg | | | | Sorafenib Equivalent, 400 mg | | |
|---|---|---|---|---|---|---|
| CBNA/ | Sorafenib, | CBNA, | Total, | Sorafenib, | CBNA, | Total, |
| Sorafenib | mg | mg | mg | mg | mg | mg |
| 1:1 | 135 | 62.5 | 197.5 | 270 | 125 | 395 |
| 2:1 | 105 | 95 | 200 | 210 | 190 | 400 |
| 3:1 | 82.5 | 116 | 198.5 | 165 | 232 | 397 |
| 4:1 | 70 | 125 | 195 | 140 | 250 | 390 |
| 5:1 | 60 | 137.5 | 197.5 | 120 | 275 | 395 |
| 6:1 | 53.5 | 146.5 | 199.5 | 107 | 293 | 400 |

Since the safety of CBNA was shown to be better than that of sorafenib (FIGS. 7 and 11), it is concluded that mixtures of sorafenib and CBNA are better candidates in treating HCC because the dosage of sorafenib could be as low as one sixth of the clinical dose of sorafenib.

### REFERENCES

The following references are indicative of the level of skill of one skilled in the art, and are incorporated herein by reference in their entirety, where permitted.
Ahn, S. W., B. Ferland and O. H. Jonas (2021). “An Interactive Pipeline for Quantitative Histopathological Analysis of Spatially Defined Drug Effects in Tumors.” J Pathol Inform 12:34.
Blasco-Benito, S., M. Seijo-Vila, M. Caro-Villalobos, I. Tundidor, C. Andradas, E. Garcia-Taboada, J. Wade, S. Smith, M. Guzman, E. Perez-Gomez, M. Gordon and C. Sanchez (2018). “Appraising the “entourage effect”: Antitumor action of a pure cannabinoid versus a botanical drug preparation in preclinical models of breast cancer.” Biochem Pharmacol 157: 285-293.
Jain, L., S. Woo, E. R. Gardner, W. L. Dahut, E. C. Kohn, S. Kummar, D. R. Mould, G. Giaccone, R. Yarchoan, J. Venitz and W. D. Figg (2011). “Population pharmacokinetic analysis of sorafenib in patients with solid tumours.” Br J Clin Pharmacol 72(2): 294-305.
Kanehisa, M., S. Goto, M. Furumichi, M. Tanabe and M. Hirakawa (2010). “KEGG for representation and analysis of molecular networks involving diseases and drugs.” Nucleic Acids Res 38 (Database issue): D355-360.
Kitisin, K., V. Packiam, J. Steel, A. Humar, T. C. Gamblin, D. A. Geller, J. W. Marsh and A. Tsung (2011). “Presentation and outcomes of hepatocellular carcinoma patients at a western centre.” HPB (Oxford) 13(10): 712-722.
Kleckner, A. S., I. R. Kleckner, C. S. Kamen, M. A. Tejani, M. C. Janelsins, G. R. Morrow and L. J. Peppone (2019). “Opportunities for cannabis in supportive care in cancer.” Ther Adv Med Oncol 11: 1758835919866362.
Meyer, P., M. Langos and R. Brenneisen (2018). “Human Pharmacokinetics and Adverse Effects of Pulmonary and Intravenous THC-CBD Formulations.” Medical Cannabis and Cannabinoids 1(1): 36-43.
Lippert, J., R. Burghaus, A. Edginton, S. Frechen, M. Karlsson, A. Kovar, T. Lehr, P. Milligan, V. Nock, S. Ramusovic, M. Riggs, S. Schaller, J. Schlender, S. Schmidt, M. Sevestre, E. Sjogren, J. Solodenko, A. Staab and D. Teutonico (2019). “Open Systems Pharmacology Community—An Open Access, Open Source, Open Science Approach to Modeling and Simulation in Pharmaceutical Sciences.” CPT Pharmacometrics Syst Pharmacol 8(12): 878-882.
Moreno, E., M. Cavic, A. Krivokuca, V. Casado and E. Canela (2019). “The Endocannabinoid System as a Target in Cancer Diseases: Are We There Yet?” Front Pharmacol 10: 339.
Torres, S., M. Lorente, F. Rodriguez-Fornes, S. Hernandez-Tiedra, M. Salazar, E. Garcia-Taboada, J. Barcia, M. Guzman and G. Velasco (2011). “A combined preclinical therapy of cannabinoids and temozolomide against glioma.” Mol Cancer Ther 10(1): 90-103.
Zhong, N. (2020). Cannabinol inhibits proliferation and induces cell cycle arrest and apotosis in glioblastoma, hepatocellular carcinoma and breast cancer cells. M.Sc., University of Lethbridge.

1. A pharmaceutical composition comprising a synergistic combination of cannabinol (CBN) or cannabinolic acid (CBNA) and sorafenib or regorafenib, in therapeutically effective amounts for treatment against cancer.

2. The composition of claim 1 wherein the combination comprises CBNA, and the molar ratio of CBNA:sorafenib or regorafenib is between about 1:1 to about 6:1.

3. The composition of claim 2 wherein the molar ratio is between about 2:1 to about 5:1.

4. The composition of claim 3 wherein the molar ratio is about 3:1 or about 4:1.

5. The composition of claim 1 wherein the combination comprises sorafenib.

6. The composition of claim 1 wherein the combination comprises regorafenib.

7. An oral dosage form comprising the composition of claim 1 and a pharmaceutically acceptable excipient.

8. A method of treating hepatocellular carcinoma, renal cell carcinoma, thyroid carcinoma, metastatic colorectal cancer, gastrointestinal stromal tumors, comprising administering a therapeutically effective amount of cannabinol (CBN) or cannabinolic acid (CBNA) and sorafenib or regorafenib to a subject in need, in a fixed combination or a non-fixed combination.

9. The method of claim 8 wherein the cancer is a hepatocellular carcinoma, renal cell carcinoma, and thyroid carcinoma.

10. Use of a synergistic combination of CBN or CBNA and sorafenib or regorafenib to treat a cancer which is hepatocellular carcinoma, renal cell carcinoma, thyroid carcinoma, metastatic colorectal cancer, or gastrointestinal stromal tumors.

11. The use of claim 10, wherein the molar ratio of CBNA:sorafenib or regorafenib is between about 1:1 to about 6:1.

12. The use of claim 11 wherein the molar ratio is between about 2:1 to about 5:1.

13. The use of claim 12 wherein the molar ratio is about 3:1 or about 4:1.

14. The use of claim 10, wherein the cancer is hepatocellular carcinoma, renal cell carcinoma, thyroid carcinoma, metastatic colorectal cancer, or gastrointestinal stromal tumors.

15. The use of claim 10 wherein the combination is CBNA and sorafenib.

16. The use of claim 15 wherein the cancer is hepatocellular carcinoma, renal cell carcinoma, or thyroid carcinoma.

17. The use of claim 16 wherein the cancer is hepatocellular carcinoma.

18. The method of claim 8, wherein the molar ratio of CBN or CBNA to sorafenib or regorafenib is between about 1:1 to about 6:1.

19. The method of claim 18 wherein the molar ratio is between about 2:1 to about 5:1.

20. The method of claim 19 wherein the molar ratio is about 3:1 or about 4:1.
